(19) United States
(12) Patent Application Publication
(10) Pub. No.: US 2012/0294869 A1
(43) Pub. Date: Nov. 22, 2012

(54) Methods for Treating Fatty Liver Disease

(75) Inventors: Giuseppe Pizzorno, Las Vegas, NV (US); Amy Ziemba, Las Vegas, NV (US); Steve Brotman, Las Vegas, NV (US)

(73) Assignee: Nevada Cancer Institute, Las Vegas, NV (US)

(21) Appl. No.: 13/471,853

(22) Filed: May 15, 2012

Related U.S. Application Data
(60) Provisional application No. 61/486,481, filed on May 16, 2011.

(51) Int. Cl. A61K 31/7072; A61P 1/16; A61K 31/715; A61K 48/00; A61K 39/395; A61K 38/02

(52) U.S. Cl. 424/158.1; 514/50; 514/1.1; 514/54; 514/44 R

(57) ABSTRACT

The present invention relates to the compositions, formulations and methods of treating fatty liver disorders, such as non-alcoholic fatty liver disease (NAFLD) and non-alcoholic steatohepatitis (NASH) and their sequelae by administration of uridine or a compound that modulates one or more uridine phosphorylases in a subject in need thereof.

### CROSS REFERENCE TO RELATED APPLICATIONS

This application claims the benefit of U.S. Provisional Application No. 61/486,481, filed May 16, 2011.

### INCORPORATION BY REFERENCE

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### BACKGROUND OF THE INVENTION

1. Field of the Invention

The invention pertains to the compositions, formulations and methods of treating fatty liver disorders, such as non-alcoholic fatty liver disease (NAFLD) and non-alcoholic steatohepatitis (NASH) and their sequelae by administration of uridine or other means to elevate uridine concentrations in a subject in need thereof. In particular, the invention relates to modulating uridine levels in subjects suffering from FLD and/or complications of FLD reduce fatty deposits in the liver to treat or prevent FLD and its associated complications.

2. Background Information

Obesity, metabolic syndrome, type 2 diabetes, and atherosclerosis are increasing at an alarming rate in the Western world. In recent years, fatty liver has emerged as an independent risk factor for these diseases. Fatty liver is the accumulation of triglycerides and other fats within hepatocytes. Fatty liver disease can range from fatty liver alone (also known as “steatosis”), to fatty liver associated with inflammation or steatohepatitis. Non-alcoholic fatty liver disease (NAFLD) and non-alcoholic steatohepatitis are the most common causes of chronic liver disease in the adult population and represents a crucial risk factor for progression to liver failure, cirrhosis and hepatocellular carcinoma. While steatosis affects approximately 30% of the population, 80% of obese patients have NAFLD and 50% of patients undergoing bariatric surgery have steatohepatitis. NAFLD also represents the most common cause of liver disease in children. It is estimated that NAFLD affects up to 20 percent of adults and nearly 5 percent of children. Some experts estimate that about two thirds of obese adults and half of obese children may have fatty liver. In the past ten years the rate of obesity in our country has doubled in adults and tripled in children and teenagers, which may explain why NAFLD and NASH are becoming more common. NASH can cause scarring and hardening of the liver, leading to cirrhosis, a very serious disease that may require a liver transplant, and eventually to hepatocellular carcinoma. Because of rising rates of obesity, NASH has become increasingly common. Some estimates suggest that one-third of adult Americans are affected, and this is consistent with the fact that one-third of Americans are considered obese.

There is no established medical treatment for fatty liver. Presently, treatment of NAFLD is limited to 1) treatment of associated metabolic disorders such as diabetes and hyperlipidemia; 2) the management of insulin resistance focusing on weight loss, exercise and/or a pharmacological approach; and 3) the use of antioxidants as hepatic protection agents. Despite the use of many different therapeutic modalities, no clear treatment is currently available to address NAFLD. Because it is clinically important to resolve NAFLD and its sequelae, new approaches aimed at preventing and reversing fat accumulation in the liver are necessary.

Accordingly, there exists a need for compositions and methods for treating fatty liver diseases.

### SUMMARY OF THE INVENTION

The present invention relates to compositions and methods for treating fatty liver disorders, including non-alcoholic fatty liver disease (NAFLD) and non-alcoholic steatohepatitis (NASH), by administration of a modulator of uridine phosphorylase, such as, for example, uridine or a compound that affects the bioavailability or circulating levels of uridine and/or modulate the activity or expression of uridine phosphorylases in a subject in need thereof.

Accordingly, in one aspect of the present invention, a method of treating a fatty liver disorder in a subject is provided, comprising administering to the subject a therapeutically effective amount of a uridine phosphorylase (UPP) modulator.

In one embodiment, the uridine phosphorylase is UPP-1. In another embodiment, the uridine phosphorylase is UPP-2. In some embodiments, the modulator is capable of targeting both UPP-1 and UPP-2. The UPP modulator can be any type of molecule. In other embodiments, the UPP modulator is uridine. In some embodiments, the UPP modulator is a small molecule compound, an antibody, a protein, polypeptide or peptide, a polysaccharide, a nucleic acid, including an inhibitory nucleic acid, an siRNA, an aptamer, or any combination thereof.

In another aspect, the present invention provides a pharmaceutical composition comprising a therapeutically effective amount of a uridine phosphorylase (UPP) modulator and a pharmaceutically acceptable carrier or diluent. The UPP modulator may be uridine.

The invention also provides formulations of uridine and methods of using these formulations for treating fatty liver disorders, including non-alcoholic fatty liver disease (NAFLD) and non-alcoholic steatohepatitis (NASH). These formulations introduce uridine to liver cells in a therapeutically relevant and bioavailable form. In contrast to over-the-counter, commercially available dietary supplements that contain uridine that typically have short half-lives, the uridine formulations of the invention allow uridine to be introduced into liver cells in a pharmaceutically and therapeutically meaningful way. In some embodiments, the formulations increase the serum half-life of uridine. In some embodiments, the formulations increase the absorption of uridine by liver cells.

In some embodiments, the methods of the present invention include the administration of a UPP modulator and/or a uridine formulation, optionally in combination with one or more additional therapeutic agents, i.e., a “co-therapy” regimen. These “co-therapies” can be administered sequentially or concurrently. One or more of the UPP modulators described herein and/or the uridine formulation with or without one or more additional therapeutic agents, can be administered to a subject, preferably a human subject, in the same pharmaceutical composition. Alternatively, the UPP modulator(s) and/or the uridine formulation and optionally one or more additional therapeutic agents, can be administered concurrently, separately or sequentially to a subject in separate pharmaceutical compositions. The UPP modulators, the uridine formulation, and the one or more additional therapeutic agents may be administered to a subject by the same or different routes of administration. In some embodiments, the UPP modulators, the uridine formulation and optional additional therapeutic agents are capable of functioning together to have an additive or synergistic effect.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from the following detailed description and claims.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1: (A) shows representative CARS microscopy images of liver tissues of wild-type (WT), UPP-1-knockout (KO) and UPP-1-conditional knock-in (TG) mice. Images are presented as a three-dimensional stack of 25 frames taken at 1-micron intervals along the vertical axis. (B) and (C) are bar graphs representing a quantitative analysis of lipid-droplet (LD) number (B) and size (C) in each hepatocyte. Error bars represent the standard deviations across 200 cells analyzed. Three mice per animal group were used for analysis.

FIG. 2 is a graph showing serum concentrations of triglyceride in fasted mice expressing different levels of UPP-1. Blood samples for serum triglycerides concentrations were collected by retro-orbital bleeding after a 4 hour fasting. Serum was collected and triglycerides determined with Wako Diagnostic L-Type TG M kit.

FIG. 3: (A) shows representative CARS microscopy images of liver tissues from WT and UPP-1-TG mice. Images are presented as 3-D stacks. (B) is a bar graph representing quantitative analysis of liver lipid in 18 probed volumes with xyz dimensions of 125×125×25 μm. Three mice per group were used for analysis. Lipid level is defined as the square root of the CARS signal arising from the lipid droplets. Lipid level is normalized to 1 for WT mice. Error bars represent the standard deviations. (C) is a graph depicting Raman spectroscopy analysis of lipid droplet composition.

FIG. 4: (A) shows Raman spectra of 3 fatty acid methyl esters, stearate C18:0, oleate C18:1, and linoleate C18:2. Red and blue arrows point to 1445 cm⁻¹ and 1660 cm⁻¹ peaks, respectively. (B) is a graph showing that 11660/11445 is linearly dependent on lipid-chain unsaturation. (C) is a bar graph depicting lipid-chain unsaturation of liver lipid droplets as a function of WT and UPP-1-TG mice. Error bars represent standard deviation across 18 lipid droplets measured.

FIG. 5: (A) and (B) are bar graphs showing the effect of low-fat (10%) and high-fat (45%) diets on the weight of the three different mouse strains after 4 weeks. Mice (6 per group) were weighed twice a week. Data are expressed as percent change over initial weight.

FIG. 6: (A) shows representative CARS images of isolated primary hepatocytes from WT and UPP-1-TG mice. (B) is a bar graph representing quantitative analysis of intracellular lipid level in 15 isolated hepatocytes from each group. Intracellular lipid level is defined as the square root of the CARS signal arising from the intracellular lipid droplets of each hepatocyte. Intracellular lipid level is normalized to 1 for WT hepatocytes.

FIG. 7 depicts experiments tracking de novo lipid synthesis and fatty acid uptake with 13C glucose and ²H palmitic acid. (A) shows lipid droplet accumulation due to de novo synthesis of lipids in 3T3-L1 cells. (B) depicts lipid droplet accumulation due to exogenous deuterated palmitic acid. Note the prominent C₂H₂ peak at 2150 cm⁻¹. (C) depicts lipid droplet accumulation due to both de novo lipid synthesis and uptake of exogenous deuterated palmitic acid. (D) shows lipid droplet accumulation due to both de novo lipid synthesis using ¹³C glucose and uptake of exogenous deuterated palmitic acid. Inset: Note the distinctive ¹³C-¹³C peak at 1600 cm⁻¹ and 13C═O peak at 1710 cm⁻¹.

FIG. 8 shows UPP-2-KO mouse characterization. (A) depicts the targeted disruption of the UPP-2 locus at exon 4, wherein restriction sites and probe locations used for Southern blot analysis are indicated. (B) is a picture representing Southern blot screening of embryonic stem (ES) cells using the 5′ external probe that detects the wild-type allele (6.1 kb) and the mutated allele (9.4 kb). (C) represents PCR analysis of the ES cells using the mutant primers (24 and 25) indicated. Heterozygotes are indicated by the 339 bp PCR product, and homozygous wild-type animals (#3, 11, and 12) are negative.

FIG. 9: (A) shows a structural comparison of active UPP-2 (blue), active UPP-1 (gold) and oxidized UPP-2 (turquoise) in an inactivated conformation. The formation of an internal disulfide bridge (red) contorts a loop region of the enzyme, pulling a highly conserved arginine residue (R1 OO/R94) away from the protein's active site, where it is required for coordination of the phosphate ligand. In (B), the cysteine pair underlying this mechanism is conserved among all known mammalian UPP-2 homologues. (C) shows the observed conformational flexibility in UPP-2 creates a void near the active site that may be exploitable for discovery of selective small molecule inhibitors of this specific homologue.

### DETAILED DESCRIPTION OF THE INVENTION

Uridine phosphorylase is a key enzyme of pyrimidine salvage pathways, catalyzing the reversible phosphorolysis of ribosides of uracil to nucleobases and ribose-1-phosphate. UPP-2 is a lipid-regulated, liver-specific uridine phosphorylase and is highly homologous to uridine phosphorylase I (UPP-1, EC2.4.2.3). Human UPP-2, a 317 amino acid protein of 35.6 kD molecular mass, is 60% identical to human UPP-1. In humans, the UPP-2 protein is expressed in kidney, liver and spleen while murine UPP-2 is present in liver and in much less amount in kidney and brain. UPP-2 expression in mouse liver has been found to be inhibited by the PPAR-α agonist fenofibrate and to a lesser extent, by the farnesoid X receptor (FXR) agonist chenodeoxycholic acid. However, the liver X receptor (LXR) agonist T0901317 was able to generate a potent induction of UPP-2 in mouse liver tissue (Zhang, Y., et al. (2004) Molecular Endocrinology 18: 851-862). Identification of UPP-2 suggests that there is a previously unsuspected link between lipid and uridine metabolism.

The substrate for both uridine phosphorylases, uridine, is an important nucleoside precursor in the pyrimidine salvage pathway (Traut, T. W., and Jones, M. E. (1996) Prog. Nucleic Acids Res. Mol. Biol. 53: 1-78; Grem, J. L. (2000) Investig. New Drugs 18: 299-313; Lucas, Z. J. (1967) Science 156: 1237-1240). Liver, along with erythrocytes and kidney, maintains de novo pyrimidine biosynthesis and supplies other tissues with uridine for salvage. Most normal tissues in adults rely on the salvage of uridine from plasma (Sladek, F. M., Hepatocyte nuclear factor 4. In: Tranche, F., Yaniv, M., eds. Liver gene expression. (1994) Austin, Tex.: R. G. Landes Co.; 207-230). Uridine also participates in the regulation of several physiological and pathological processes (Wice, B. M., and Kennell, D. (1982) J. Biol. Chem. 257: 2578-2583; Becroft, D. M., et al. (1969) J. Pediatr. 75: 885-891; Dagani, F., et al. (1984) Neurochem. Res. 9, 1085-1099; Page, T., et al. (1997) Proc. Natl. Acad. Sci. U.S.A. 94, 11601-11606; Darnowski, J. W., et al. (1991) Biochem. Pharmacol. 41, 2031-2036). In the absence of sugar, uridine can serve as an essential precursor for both carbohydrate metabolism and nucleic acid synthesis.

The present inventors have unveiled the role of uridine and liver uridine phosphorylase (UPP-2) in modulating the accumulation and metabolism of triglycerides in hepatocytes. Modulation of uridine phosphorylase activity with consequent variation of plasma and tissue levels of uridine/pyrimidines results in changes in the ability of the liver to accumulate and metabolize lipids (fatty acids, triglycerides, etc.), therefore providing a new mechanism to treat fatty liver disorders.

Through the use of genetically modified mouse models with differential expression of uridine phosphorylase 1 (UPP-1) and uridine phosphorylase 2 (UPP-2), the present inventors have demonstrated that disruption of uridine homeostasis in plasma and normal tissues results in changes to the hepatic ability to metabolize and accumulate lipids, mainly in the hepatocytes. More specifically, low levels of circulating uridine cause accumulation of lipids in the hepatocytes, while high levels of uridine, endogenously produced by limiting the activity of UPPs or exogenously administered, result in a significant reduction of accumulated lipids in liver, a substantial decrease in serum triglycerides, and reduced ability to gain weight when fed a diet high in calories derived from fat.

Accordingly, the present invention relates to compositions comprising modulators of uridine phosphorylases, such as uridine, uridine prodrugs and/or compounds that increase the bioavailability or circulating levels of uridine and/or modulate the activity or expression of uridine phosphorylases. The invention also encompasses methods of treating fatty liver disorders using such compositions.

### DEFINITIONS

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

The terms “administration” and or “administering” a compound should be understood to mean providing a compound of the invention to a subject in need of treatment.

As used herein, “expression” and “expression levels” include but are not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

Fatty liver disorders, also known as fatty liver or fatty liver disease (FLD), relates to a condition where large vacuoles of triglyceride fat accumulate in liver cells via the process of steatosis, or abnormal retention of lipids within a cell. Despite having multiple causes, fatty liver is considered a single disease that occurs frequently in subjects with excessive alcohol intake and those who are obese (with or without effects of insulin resistance). The condition is also associated with other diseases that influence fat metabolism. FLD may be categorized into two separate conditions: alcoholic FLD and non-alcoholic FLD. Both conditions show micro-vesicular and macro-vesicular fatty changes at different stages of the disease. Accumulation of fat may also be accompanied by a progressive inflammation of the liver (hepatitis), called steatohepatitis. Fatty liver is also known in the art as alcoholic steatosis and non-alcoholic fatty liver disease (NAFLD), and the more severe forms as alcoholic steatohepatitis (part of alcoholic liver disease) and non-alcoholic steatohepatitis (NASH). Nonalcoholic fatty liver disease-associated cirrhosis is the most severe form of the disease and is characterized by liver inflammation that leads to scarring of the liver tissue, ultimately resulting in liver failure. “Modulating” or “modulate” in the context of the present invention means increasing, decreasing, or otherwise altering, adjusting, varying, changing, enhancing or inhibiting a biological event. Likewise, a “modulator” may be a compound, agent or drug that increases, decreases, alters, adjusts, varies, changes, enhances, or inhibits a biological event, such as phosphorolysis catalyzed by uridine phosphorylases, such as, e.g., UPP-1 and UPP-2, or expression of uridine phosphorylases. In certain embodiments of the invention, the UPP modulator is a small molecule compound, an antibody, a protein, polypeptide or peptide, a polysaccharide, a nucleic acid, including an inhibitory nucleic acid, an siRNA, an aptamer, or any combination thereof.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of fatty liver disorder. A subject can be male or female.

The term “treating” in its various grammatical forms in relation to the present invention refers to preventing (e.g., chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease. Treatment of fatty liver disorders, as used herein, refers to partially or totally inhibiting, delaying or preventing the progression of fatty liver disorders in a subject.

As used herein, the term “therapeutically effective amount” is intended to qualify a desired biological response, such as, e.g., partial or total inhibition, delay or prevention of the progression, onset, or development of fatty liver disorders (e.g., chemoprevention) in a subject.

Uridine is a nucleoside that is formed when uracil is attached to a ribose ring (also known as a ribofuranose) via a β-N₁-glycosidic bond. Uridine is available in phosphorylated form, i.e., uridine-5′-monophosphate (also known as 5′-uridylic acid and UMP), uridine 5′-monophosphate tris salt, uridine 5′-monophosphate salt dihydrate, uridine 5′-monophosphate salt solution, uridine 5′-monophosphate salt hydrate, uridine-¹³C₉, ¹⁵N₂ 5′-monophosphate sodium salt solution, uridine-¹⁵N₂ 5′-monophosphate sodium salt solution, uridine 5′-monophosphate trisodium salt hydrate, uridine-¹³C₉, ¹⁵N₂ 5′-monophosphate sodium salt solution, uridine-N₂ 5′-monophosphate sodium salt solution, uridine-5′-diphosphate (UDP), uridine 5′-diphosphate tris salt, uridine 5′-diphosphate salt dihydrate, uridine 5′-diphosphate salt solution, uridine 5′-diphosphate salt hydrate, uridine-¹³C₉, ¹⁵N₂ 5′-diphosphate sodium salt solution, uridine-¹⁵N₂ 5′-diphosphate sodium salt solution, uridine 5′-diphosphate trisodium salt hydrate, uridine-¹³C₉, ¹⁵N₂ 5′-diphosphate sodium salt solution, uridine-¹⁵N₂ 5′-diphosphate sodium salt solution, uridine-5′-triphosphate (UTP), UTPγS, MRS2498, uridine 5′-triphosphate tris salt, uridine 5′-triphosphate salt dihydrate, uridine 5′-triphosphate salt solution, uridine 5′-triphosphate salt hydrate, uridine-¹³C₉, ¹⁵N₂ 5′-triphosphate sodium salt solution, uridine-¹⁵N₂ 5′-triphosphate sodium salt solution, uridine 5′-triphosphate trisodium salt hydrate, uridine-¹³C₉, ¹⁵N₂ 5′-triphosphate sodium salt solution, uridine-¹⁵N₂ 5′-triphosphate sodium salt solution, 2-diuridine tetraphosphate, thio-UTP tetrasodium salt, denufosol tetrasodium, or UTPγS trisodium salt, prodrugs known in the art as triacetyluridine (TAU) or uridine triacetate (PN501), acyl derivatives of uridine such as those described in U.S. Pat. No. 7,582,619 (i.e., 2′,3′,5′-tri-0-pyruvyluridine), 2,2′-anhydro-5-ethyluridine, 5-ethyl-2-deoxyuridine, and acyclouridine compounds such as 5-benzyl substituted acyclouridine congeners including, e.g., benzylacyclouridine, benzyloxybenzylacyclouridine, aminomethyl-benzylacyclouridine, aminomethylbenzyloxy-benzylacyclouridine, hydroxymethyl-benzyloxy-benzylacyclouridine (see also, W089/09603 and W091/16315), and in dietary supplements such as Mitocnol and NucleomaxX, derived from sugar cane extract.

“Uridine phosphorylase” or “UPP” is an enzyme that catalyzes the reversible phosphorolysis reaction of uridine (in the presence of phosphate) to uracil and a-D-ribose-1-phosphate. UPP is a key enzyme of pyrimidine salvage pathways. Humans possess two known isoforms of UPP: UPP-1 and UPP-2. The present invention encompasses compositions and methods of modulating the activity of one or both human UPP isoforms in a subject.

Compounds and Pharmaceutical Compositions

The compounds of the present invention may exist in one or more particular geometric, optical, enantiomeric, diastereomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The compounds of the invention when used in pharmaceutical or diagnostic applications may be prepared in a racemic mixture or an essentially pure enantiomer form, with an enantiopurity of at least 90% enantiomeric excess (EE), preferably at least 95% EE, more preferably at least 98% EE, and most preferably at least 99% EE. Enantiomeric excess values provide a quantitative measure of the excess of the percentage amount of a major isomer over the percentage amount of a minor isomer which is present therewith, and may be readily determined by suitable methods well-known and established in the art, as for example chiral high pressure liquid chromatography (HPLC), chiral gas chromatography (GC), nuclear magnetic resonance (NMR) using chiral shift reagents, etc.

A “pharmaceutical composition” is a formulation containing the compounds of the present invention in a form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In general, compounds and pharmaceutical compositions of the invention may be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors involved, as readily determinable within the skill of the art. Suitable therapeutic doses of the compounds of the invention may be in the range of 1 microgram (μg) to 1000 milligrams (mg) per kilogram body weight of the recipient per day, and any increment in between, such as, e.g., 1, 2, 3, 5, 10, 25, 50, 75, 100, 200, 300, 400, 500, 600, 700, 800, 900, or 1000 μg (1 mg); 2, 3, 5, 10, 25, 50, 75, 100, 200, 300, 400, 500, 600, 700, 800, 900 or 1000 mg. A desired dose may preferably be presented as two, three, four, five, six, or more sub-doses administered at appropriate intervals throughout the day. These sub-doses may be administered in unit dosage forms, for example, containing from 1 μg to 1000 mg of active ingredient per unit dosage form. Alternatively, if the condition of the recipient so requires, the doses may be administered as a continuous infusion. The mode of administration and dosage forms will of course affect the therapeutic amounts of the compounds which are desirable and efficacious for the given treatment application.

For example, orally administered dosages typically are at least twice, e.g., 2-10 times, the dosage levels used in parenteral administration methods, for the same active ingredient. In oral administration, dosage levels for delta receptor binding compounds of the invention may be on the order of 5-200 mg/70 kg body weight/day. In tablet dosage forms, typical active agent dose levels are on the order of 10-100 mg per tablet.

The compounds of the present invention may be administered per se as well as in the form of pharmaceutically acceptable esters, salts, and ethers, as well as other physiologically functional derivatives of such compounds. Compounds of the invention may be amorphous or polymorphic. The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Examples of crystal lattice forms include, but are not limited to, cubic, isometric, tetragonal, orthorhombic, hexagonal, trigonal, triclinic, and monoclinic. Recrystallization solvent, rate of crystallization, storage temperature, and other factors may cause one crystal form to dominate. Crystal polymorphs of the compounds can be prepared by crystallization under different conditions.

Additionally, the compounds of the present invention, for example, the salts of the compounds, can exist in either hydrated or unhydrated (the anhydrous) form or as solvates with other solvent molecules. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H20.

Non-limiting examples of hydrates include monohydrates, dihydrates, etc. Non-limiting examples of solvates include ethanol solvates, acetone solvates, etc.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like.

Exemplary organic bases are isopropylamine, diethylamine, ethanolamine, trimethylamine, dicyclohexylamine, choline, and caffeine.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form or in inhaled forms. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods.

For example, oral compositions can be tablets or gelatin capsules comprising the active ingredient together with a pharmaceutically acceptable carrier, including any one or a combination of the following components: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners.

Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances.

Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Techniques for formulation and administration of the disclosed compounds of the invention can be found in Remington: the Science and Practice of Pharmacy, 19th edition, Mack Publishing Co., Easton, Pa. (1995).

Compounds of the invention can be administered in therapeutically effective amounts in combination with one or more additional therapeutic agents as defined herein. For example, synergistic effects can occur with other substances used in the treatment of cardiovascular disease (including atherosclerosis, cardiomyopathy or myocarditis, congestive heart failure, coronary artery disease, peripheral artery disease, arrhythmia, ischemia), obesity, insulin resistance, Metabolic Syndrome, Type I and/or Type II diabetes mellitus, hypertension, or other related diseases. Where the compounds of the invention are administered in conjunction with other therapies, dosages of the co-administered compounds will of course vary depending on the type of co-drug employed, on the specific drug employed, on the condition being treated and so forth.

As used herein, the terms “combination treatment”, “combination therapy”, “combined treatment” or “combinatorial treatment”, used interchangeably, refer to a treatment of an individual with at least two different therapeutic agents. The terms “co-administration” or “combined administration” or the like as utilized herein are meant to encompass administration of the selected therapeutic agents to a single patient, and are intended to include treatment regimens in which the agents are not necessarily administered by the same route of administration or at the same time. The term “pharmaceutical combination” means a product that results from the mixing or combining of more than one active ingredient and includes both fixed and non-fixed combinations of the active ingredients. A “fixed combination” means that the active ingredients, e.g. a compound as disclosed herein and one or more additional therapeutic agents, are both administered to a patient simultaneously in the form of a single entity or dosage. A “non-fixed combination” means that the active ingredients, e.g. a compound as disclosed herein and one or more additional therapeutic agents, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

As used herein, the uridine formulations, UPP modulators, or other compounds of the present invention may be administered with one or more additional therapeutic agents, such as, without limitation, agents for pulmonary hypertension, such as ambrisentan, bosentan, treprostinil, sildenafil, epoprostenol, treprostenol, iloprost, aldosterone receptor antagonists like spironolactone and eplerenone, angiotensin-converting enzyme inhibitors such as trandolapril, fosinopril, enalapril, captopril, ramipril, moexipril, lisinopril, quinapril, benazepril, and perindopril, angiotensin II inhibitors such as eprosartan, olmesmian, telmismian, losartan, valsmian, candesartan, and irbesmian, anti-anginal agents like nitroglycerin, isosorbide mononitrate, and isosorbide dinitrate, anti-arrhythmic agents including moricizine, quinidine, disopyramide, phenyloin, propafenone, flecamide, mexilitene, lidocaine, procainamide, propranolol, acebutolol, amiodarone, dofetilide, dronedarone, sotalol, ibutilide, diltiazem, verapamil, nifedipine, nimodipine, felodipine, nicardipine, clevidipine, isradipine, bepridil, nisoldipine, adenosine, and digoxin, P-adrenergic receptor antagonists like betaxolol, bisoprolol, metoprolol, atenolol, nebivolol, nadolol, carvedilol, labetalol, timolol, carteolol, penbutolol, pindolol, and esmolol, anti-diabetic agents including secretagogues such as sulfonylurea, tolbutamide, acetohexamide, tolazamide, chlorpropamide, glipizide, glyburide, glimepiride, glibenclamide, gliclazide, meglitinide such as nateglinide, senaglinide, repaglinide, insulin sensitizers such as biguanides, metformin, thiazolidinediones such as rosiglitazone, isaglitazone, darglitazone, englitazone, and pioglitazone, a-glucosidase inhibitors such as miglitol, voglibose, emiglitate, and acarbose, glucagon-like peptide analogs and agonists such as exenatide, liraglutide, and taspglutide, dipeptidyl peptidase-4 inhibitors like vildagliptin, sitagliptin, and saxagliptin, amylin analogs such as pramlintide, ligands or agonists of peroxisome proliferator activated receptor (PPAR)-α, β, δ, and γ cholesterol-lowering agents such as hydroxymethylglutaryl-Coenzyme A (HMG-CoA) reductase inhibitors like statins, such as, e.g., atorvastatin, fluvastatin, lovastatin, pitavastatin, pravastatin, rosuvastatin, and simvastatin, agonists of retinoid X receptors (RXR) such as, e.g., ALRT-268, LG-1268, or LG-1069, glucokinase activators, inhibitors of hepatic enzymes involved in stimulation of gluconeogenesis and/or glycogenolysis, diuretics such as acetazolamide, dichlorphenamide, methazolamide, torsemide, furosemide, bumetanide, ethacrynic acid, amiloride, triamterene, indapamide, metolazone, methylclothiazide, hydrochlorothiazide, chlorothiazide, metolazone, bendroflumethiazide, polythiazide, and chlorthalidone, vasodilators like alprostadil, hydralazine, minoxidil, nesiritide, and nitroprusside, and other anti-lipidemic agents like cholestyramine, colestipol, clofibrate, gemfibrozil, probucol or dextrothyroxine.

Methods of Screening for Compounds and Compositions

Methods for screening and identifying a candidate test compound for treating a fatty liver disorder may comprise, for example, contacting one or more isoforms of a uridine phosphorylase protein with a test compound; and determining whether the test compound interacts with the uridine phosphorylase protein, wherein a compound that interacts with the uridine phosphorylase is identified as a candidate compound for treating a fatty liver disorder. Compounds suitable for therapeutic testing may be screened initially by identifying compounds which interact with one or more UPP isoforms (e.g., UPP-1 and UPP-2). By way of example, screening might include recombinantly expressing one or more UPP proteins of this invention, purifying the proteins, and affixing the proteins to a substrate. Test compounds can then be contacted with the substrate, typically in aqueous conditions, and interactions between the test compound and the UPP protein are measured, for example, by measuring elution rates as a function of salt concentration.

Certain proteins may recognize and interact with one or more UPP proteins, in which case the UPP proteins may be detected by, e.g., immunoprecipitation and immunoblotting.

The ability of a test compound to modulate the activity of one or more UPP proteins may be measured. The techniques used to measure the activity of a UPP protein will vary depending on the function and properties of the biomarker. For example, an enzymatic activity of a UPP protein may be assayed with a radiolabeled uridine molecule and the output of the product, uracil and α-D-ribose-1-phosphate, can be readily measured. The ability of potentially therapeutic test compounds to inhibit or enhance the activity of a UPP protein may be determined by measuring the rates of catalysis in the presence or absence of the test compounds. The ability of a test compound to interfere with a non-enzymatic (e.g., structural) function or activity of a UPP protein may also be measured. For example, the self-assembly of a multi-protein complex which includes one or more UPP proteins may be monitored by spectroscopy in the presence or absence of a test compound.

Test compounds capable of modulating the activity of any of the UPP proteins of this invention may be administered to subjects who are suffering from or are at risk of developing a fatty liver disorder. For example, the administration of a test compound which increases the activity of one or more UPP proteins may decrease the risk of a fatty liver disorder in a subject if the activity of the UPP proteins in vivo prevents the onset or progression a fatty liver disorder.

Conversely, the administration of a test compound which decreases the activity of one or more UPP proteins may decrease the risk of a fatty liver disorder in a subject if the increased activity of the UPP proteins is responsible, at least in part, for the onset or progression of a fatty liver disorder.

At the clinical level, screening a test compound includes obtaining samples from test subjects before and after exposure to a test compound. The levels in the samples of one or more of UPP proteins may be measured and analyzed to determine whether the levels of the UPP proteins change after exposure to a test compound. The samples may be analyzed by any appropriate means known to one of skill in the art. For example, the levels of one or more of the biomarkers of this invention may be measured directly by Western blot using radio- or fluorescently-labeled antibodies which specifically bind to the biomarkers. Alternatively, changes in the levels of mRNA encoding the one or more UPP proteins may be measured and correlated with the administration of a given test compound to a subject. In a further embodiment, the changes in the level of expression of one or more of UPP proteins may be measured using in vitro methods and materials. For example, human tissue cultured cells which express, or are capable of expressing, one or more of UPP proteins may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with a fatty liver disorder, test compounds will be screened for their ability to slow or stop the progression of the disease.

Methods of identifying therapeutic targets for a fatty liver disorder generally comprise comparing an expression profile of a cell isolated from a subject known to have the fatty liver disorder with an expression profile of a reference cell, wherein the comparison is capable of classifying proteins or transcripts in the profile as being associated with a fatty liver disorder. Reference cells may be normal cells (e.g., liver cells that are not derived from a subject known to have a fatty liver disorder) or cells a different stage of the fatty liver disorder from the cells being compared to. The reference cells may be primary cultured cells, freshly isolated cells, established cell lines or other cells determined to be appropriate to one of skill in the mi. Transcripts and proteins associated with a fatty liver disorder include cells that differentiate between the states or stages of a fatty liver disorder and between normal and cell lines derived from subjects having a fatty liver disorder. The transcripts and proteins may also differentiate between different types or levels of severity of a fatty liver disorder. The proteins may be secreted proteins, such that they are easily detectable from a blood sample or biopsy. The cells may be derived from an animal model of a fatty liver disorder, such as transgenic mice lacking UPP-1, UPP-2, or both UPP-1 and UPP-2.

The subjects may be subjects who have been determined to have a high risk of a fatty liver disorder based on their family history, a previous treatment, subjects with physical symptoms known to be associated with a fatty liver disorder (including those having associated diseases, such as diabetes mellitus, obesity, etc.), subjects identified through screening assays (e.g., routine screening for a fatty liver disorder) or other techniques. Other subjects include subjects who have a fatty liver disorder and the test is being used to determine the effectiveness of therapy or treatment they are receiving. Also, subjects could include healthy people who are having a test as part of a routine examination. Samples may be collected from subjects who had been diagnosed with a fatty liver disorder and received treatment to eliminate the fatty liver disorder, or who are in remission.

Biologic Agents

The invention encompasses the use of biologic agents or therapies, such as, without limitation, inhibitory nucleic acids, small interfering RNA (sRNA), catalytic RNA and ribozymes, peptide nucleic acids (PNA), proteins, polypeptides, and peptides, antibodies, and aptamers (DNA, RNA, or peptide aptamers). Biologic agents may be designed based on the sequences of one or more uridine phosphorylases as defined herein, either in whole or in part (i.e., sequences of conserved domains), or may be designed based on identification of biomarkers that indicate disease status and progression of disease in a subject. In certain preferred examples, the invention features uridine phosphorylase inhibitory nucleic acid molecules. Uridine phosphorylase inhibitory nucleic acid molecules are essentially oligomers or oligonucleotides that may be employed as single-stranded or double-stranded nucleic acid molecule to decrease or ablate uridine phosphorylase expression.

In one approach, the uridine phosphorylase inhibitory nucleic acid molecule is a double-stranded RNA used for RNA interference (RNAi)-mediated knock-down of uridine phosphorylase gene expression. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleotides. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleotides) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al., (2002) Science 296: 550-553; Paddison et al., (2002) Genes & Devel. 16: 948-958. Paul et al., (2002) Nature Biotechnol. 20: 505-508; Sui et al., (2002) Proc. Natl. Acad. Sci. USA 99: 5515-5520; Yu et al., (2002) Proc. Natl. Acad. Sci. USA 99: 6047-6052; Miyagishi et al., (2002) Nature Biotechnol. 20: 497-500; and Lee et al., (2002) Nature Biotechnol. 20: 500-505, each of which is hereby incorporated by reference.

An inhibitory nucleic acid molecule that “corresponds” to one or more uridine phosphorylase genes comprises at least a fragment of the double-stranded gene, such that each strand of the double-stranded inhibitory nucleic acid molecule is capable of binding to the complementary strand of a target uridine phosphorylase gene. The inhibitory nucleic acid molecule need not have perfect correspondence to the reference uridine phosphorylase sequence. In one embodiment, a siRNA has at least about 85%, 90%, 95%, 96%, 97%, 98%, or even 99% sequence identity with the target nucleic acid. For example, a 19 base pair duplex having a I-2 base pair mismatch is considered useful in the methods of the invention. In other embodiments, the nucleotide sequence of the inhibitory nucleic acid molecule exhibits 1, 2, 3, 4, 5 or more mismatches. The inhibitory nucleic acid molecules provided by the invention are not limited to siRNAs, but include any nucleic acid molecule sufficient to decrease the expression of a uridine phosphorylase nucleic acid molecule or polypeptide. Each of the DNA sequences provided herein may be used, for example, in the discovery and development of therapeutic antisense nucleic acid molecule to decrease the expression of one or more uridine phosphorylases.

The invention further provides catalytic RNA molecules or ribozymes. Such catalytic RNA molecules can be used to inhibit expression of a uridine phosphorylase nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., (1998) Nature 334:585-591 and U.S. Patent Application Publication No. 20030003469, each of which is incorporated by reference. In various embodiments of this invention, the catalytic nucleic acid molecule is formed in a hammerhead or hairpin motif. Examples of such hammerhead motifs are described by Rossi et al., AIDS Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. patent application Ser. No. 07/247,100 filed Sep. 20, 1988, Hampel and Tritz, (1989) Biochemistry 28: 4929, and Hampel et al., (1990) Nucl. Acids Res. 18: 299. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. After a subject is diagnosed as having a fatty liver disorder, or at risk for recurrence of a fatty liver disorder, a method of treatment is selected.

The inhibitory nucleic acid molecules of the invention may be administered systemically in dosages between about 1 and 100 mg/kg (e.g., 1, 5, 10, 20, 25, 50, 75, and 100 mg/kg). The liver disorder can receive a dosage between about 50 and 300 mg/m²/day (e.g., 50, 75, 100, 125, 150, 175, 200, 250, 275, and 300). The amounts of the inhibitory nucleic acid molecules administered to the subject will depend, of course, on whether it is administered alone or in combination with another additional therapeutic agent, such as the uridine formulations and/or uridine phosphorylase modulator compounds disclosed herein.

One type of inhibitory nucleic acid molecule is based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC50. This modification also increases the nuclease resistance of the modified oligonucleotide. It is understood that the methods and reagents of the present invention may be used in conjunction with any technologies known to those skilled in the art that may be developed to enhance the stability or efficacy of an inhibitory nucleic acid molecule.

Inhibitory nucleic acid molecules include oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be oligomers. Oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Oligomers may also contain one or more substituted sugar moieties. Such modifications include 2′-0-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or oligomer, particularly the 3′ position of the sugar on the 3′ terminal nucleotide. Oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety. In other oligomers, both the sugar and the internucleoside linkage, i.e., the backbone, are replaced with novel groups. The nucleotide units are maintained for hybridization with a uridine phosphorylase nucleic acid molecule. Methods for making and using these oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082, 5,714,331, and 5,719,262, each of which are herein incorporated by reference. Further teaching of PNA compounds can be found in Nielsen et al., Science, 1991, 254, 1497-1500.

The invention also concerns the use of proteins, polypeptides, peptides, peptidomimetics, or antibodies that can be used as modulators of uridine phosphorylases. The term “protein” as used herein means isolated naturally occurring polypeptides, recombinantly produced proteins. Means for preparing such proteins are well understood in the art. Proteins may be in the form of the secreted protein, including truncated or mature forms. Proteins may optionally be modified to include an additional amino acid sequence which contains secretory or leader sequences, pro-sequences, sequences which aid in purification, such as multiple histidine residues, or an additional sequence for stability during recombinant production. The proteins of the present invention are preferably provided in an isolated form, and preferably are substantially purified. A recombinantly produced version of a protein, including the secreted protein, can be substantially purified using techniques described herein or otherwise known in the art, such as, for example, by the one-step method described in Smith et al, Gene, 67:31-40 (1988). Proteins of the invention also can be purified from natural, synthetic or recombinant sources using techniques described herein or otherwise known in the art.

As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)2, and Fab fragments which lack the Fe fragment of intact antibody, clear more rapidly from the circulation, and may have less non-specific tissue binding of an intact antibody (Wahl et al., (1983) J. Nucl. Med. 24:316-325. The antibodies of the invention comprise whole native antibodies, bispecific antibodies; chimeric antibodies; Fab, Fab′, single chain V region fragments (scFv) and fusion polypeptides. Antibodies include monoclonal antibodies and polyclonal antibodies. “Humanized” antibodies are antibodies in which at least pati of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

The antibodies can be delectably labeled, for example, with a radioisotope, a bioluminescent compound, a chemiluminescent compound, a fluorescent compound, a metal chelate, or an enzyme (e.g. horseradish peroxidase, alkaline phosphatase, beta-galactosidase, malate dehydrogenase, glucose oxidase, urease, catalase etc.) which, in turn, when later exposed to a substrate will react to the substrate in such a manner as to produce a chemical moiety which can be detected. The antibodies can also be immobilized on an insoluble carrier, e.g. glass, polystyrene, polypropylene, polyethylene, dextran, nylon, natural and modified celluloses, polyacrylamides, agarose and magnetic beads.

### EXAMPLES

Hepatic accumulation of lipids was examined in three distinct animal models with altered uridine metabolism: a uridine phosphorylase 1 (UPP-1) knockout mouse (Cao, D., et al. (2005) J. Biol. Chem. 280: 21169-21175); a UPP-1 and thymidine phosphorylase (TPase) double knockout, expressing UPP-2 as the only phosphorolytic activity (Lopez, L. C., et al. (2009) Human Mol. Gen. 18: 714-722); and a UPP-1 transgenic mouse model with ubiquitous over-expression of UPP-1 activity. The animal models indicated a clear link between circulating uridine concentrations and plasma triglyceride levels, as well as different effects of high-fat diet on the weight and liver lipid accumulation of various mouse strains with altered UPP-1 expression.

Examples are provided below to further illustrate different features of the present invention. The examples also illustrate useful methodology for practicing the invention. These examples do not limit the claimed invention.

### Example 1

### Generation and Characterization of UPP-1 Knockout and Transgenic Mice

The UPP-1-KO mouse was created by replacing a 2.5 kb fragment of the UPP-1 gene (including exons 4 and 5) with a 1.6 kb neomycin resistance expression cassette. See, e.g., Cao, D. et al. (2002) Cancer Res. 62: 2313-2317 and Cao, D. et al. (2005) J. Biol. Chem. 280: 21169-21175. A three to four-fold increase in the circulating plasma level of uridine (Urd) was observed in UPP-1-KO mice. The absence of UPP-1 phosphorolytic (UPase) activity was confirmed by determining the fate of a tracer dose (25 μCi/mouse) of [³H]Urd injected intraperitoneally (i.p.) in mice. A very rapid disappearance of [³H]Urd from the plasma of wild-type (WT) mice was observed, with a t_{1/2}, of approximately 3 minutes due to active phosphorolysis. In contrast, [³H]Urd t_{1/2}, was approximately 15-18 minutes in UPP-1-KO mice.

The abrogation of UPase activity in tissues has not only resulted in dramatic changes in Urd metabolism but also a major alteration in its plasma and tissue accumulation and distribution (Table 1).

TABLE 1
Plasma and tissue uridine levels

| | Plasma | Gut | Kidney | Liver | Spleen |
|---|---|---|---|---|---|
| | (μM) | (μM) | (μM) | (μM) | (μM) |
| WT | 2.5 ± 0.5 | 29.2 ± 3.3 | 24.5 ± 3.5 | 6.4 ± 2.5 | 34.9 ± 1.1 |
| UPP- | 7.2 ± 2.4 | 89.3 ± 2.3 | 71.0 ± 12.6 | 42.8 ± 7.0 | 75.2 ± 12.7 |
| 1-KO | | | | | |
| UPP- | 0.2 ± 0.1 | 9.0 ± 1.5 | 7.1 ± 2.7 | 0.5 ± 0.3 | 0.6 ± 0.2 |
| 1-TG | | | | | |

The concentration of URD in plasma has increased from 2-3 μM in the control animals to approximately 7-10 μM in UPP-1-KO mice. More importantly, this higher level of circulating pyrimidine nucleoside has caused a dramatic accumulation of URD in all of the major tissues investigated due to the activity of the Urd Na⁺-dependent active transporters. The disruption of Urd homeostasis results in changes in the size of the deoxyribonucleotide pools that are more dramatic than the ribonucleotides. Overall, the dTTP pools appear to be the most affected by UPP-1 nullification. Surprisingly, major changes in the level of purine deoxynucleotides were observed as well, possibly indicating a feedback regulatory mechanism to balance their supply for DNA synthesis (Lopez, L. C., et al. (2009) Human Mol. Gen. 18: 714-722).

To maximize the metabolism of URD, a conditional UPP-1-knock-in mouse model targeted at the ROSA26 chromosomal locus was generated (Soriano, P. (1999) Nature Genet. 21: 70-71). This model was used to create a ubiquitous UPP-1 transgenic mouse (UPP-1-TG) to reduce circulating Urd concentration in every body-compartment. The targeting construct contains the UPP-1 coding sequence driven by the CAGGS promoter, a hybrid chicken β-actin and cytomegalovirus promoter that is active in almost all tissues in vivo (Okabe M. et al. (1997) FEBS Letters. 407: 313-319). The promoter and UPP-1 coding sequence are interposed by a neomycin resistance cassette, which is flanked by loxP sites. Embryonic stem (ES) cells were transfected with the targeting construct (Animal Genomics Services at Yale University School of Medicine) and assessed for homologous recombination. Germline transmission was confirmed in chimeric mice after blastocyst injection, and the chimeric mice were bred to homozygosity. Gene expression was “knocked-in” by crossing these transgenic mice with mice expressing Cre-recombinase in various tissues of interest. Cre-recombinase activity excised the loxP sites and released a neomycin resistance cassette allowing CAGGS-driven expression of UPP-1. These mice were crossed with FVB/N-Tg(ACTB-cre)₂Mrt/J mice (Jackson Laboratories), in which Cre expression is driven by the ubiquitous β-actin promoter. Overexpression of UPP-1 was evaluated by real-time PCR and Western blot analysis, as well as determining the enzymatic activity (Table 2).

TABLE 2
Tissue uridine phosphorolytic activity

UTPase activity
(nmol/hr/mg)

| | WT | UPP-1 TG |
|---|---|---|
| Lung | 37.4 ± 4.7 | 1660 |
| Muscle | 5.6 ± 1.5 | ND |
| Spleen | 13.0 ± 3.0 | ND |
| Small Intestine | 696.8 ± 80.0 | 3480 |
| Kidney | 32.6 ± 12.4 | 441 |
| Liver | 10.7 ± 0.3 | 252 |

Quantitative RT-PCR evaluation of UPP-1 expression in the liver of UPP-1-TG mice revealed an approximately 1,000-fold increased expression of the transgene compared to WT tissue. Enzymatic activity in liver was 10.7±0.3 nmol/hr/mg for the WT animals compared to 252±68 nmol/hr/mg for the transgenic. In WT mice, a tracer dose of [³H]Urd (25 μCi, i.p.) was rapidly degraded with a t_{1/2}, of approximately 4 minutes. In UPP-1-TG mice the [³H]Urd t_{1/2}, was calculated to be less than 30 seconds, with no [³H]Urd detectable 15 minutes after administration. Table 1 summarizes the concentrations of URD in WT C57BL/6 mice, the UPP-1-KO and the UPP-1-TG models.

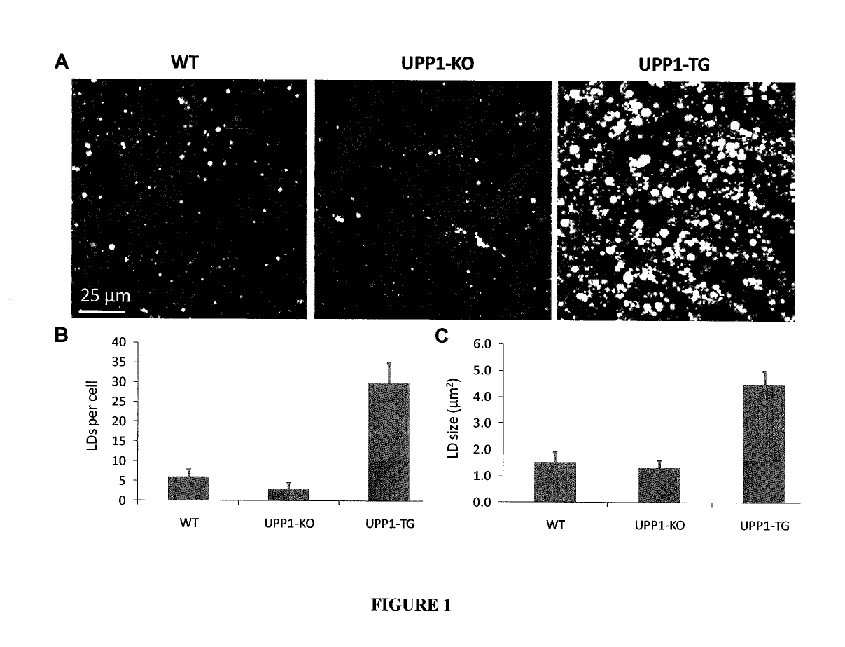

Using CARS microscopy to image 100-micron thick sections of explanted liver tissues, the effect of the disruption of URD homeostasis on liver lipids was evaluated. As reprinted in FIG. 1, a dramatic difference in liver lipid accumulation among WT, UPP-1-KO and UPP-1-TG mice was observed. The UPP-1-TG mice showed a 6-7 fold increase in the number of lipid droplets compared to WT animals and almost a 10-fold increase over the UPP-1-KO mice. In addition, the size of the lipid droplets was significantly elevated in the liver of UPP-1-TG mice with a 3-fold increase compared to the other two strains. The number of lipid droplets was reduced in UPP-1-KO mice compared to WT mice, but the size reduction did not reach significance. To confirm the results obtained through CARS microscopy, Oil Red O staining (specific for neutral triglycerides and lipids) of the frozen liver sections was conducted, which yielded similar results to the CARS technology (data not shown).

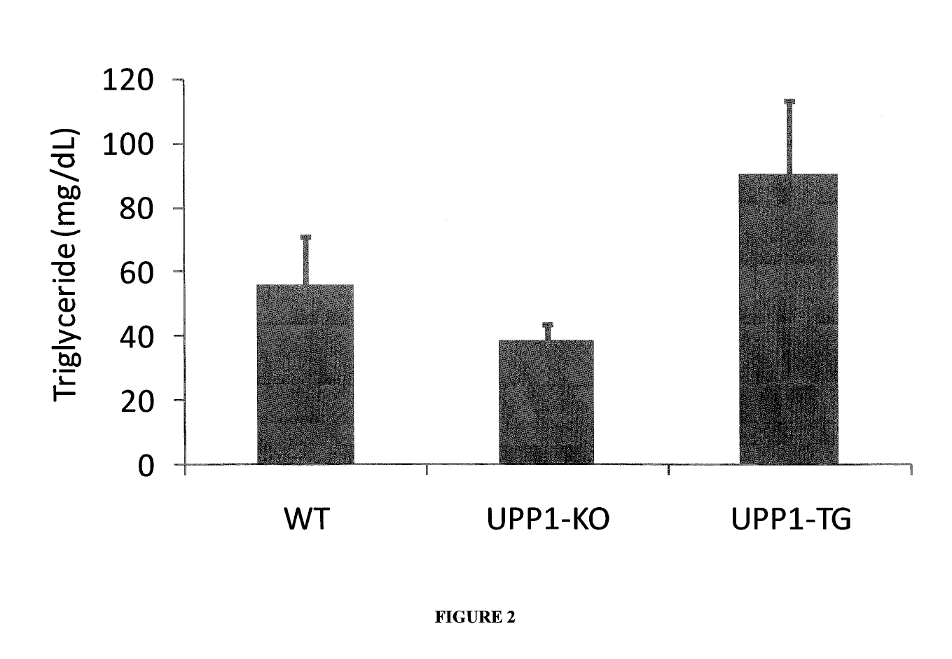

To evaluate if the changes seen in the UPP-1-TG mice were only limited to the liver, the concentration of triglycerides in serum was examined utilizing the Wako Diagnostic L-Type TG M kit. As indicated in FIG. 2, triglyceride levels were consistently 2-fold higher in serum of UPP-1-TG mice compared to the two other strains. This was true after 4 hour fasting as well as under normal feeding conditions.

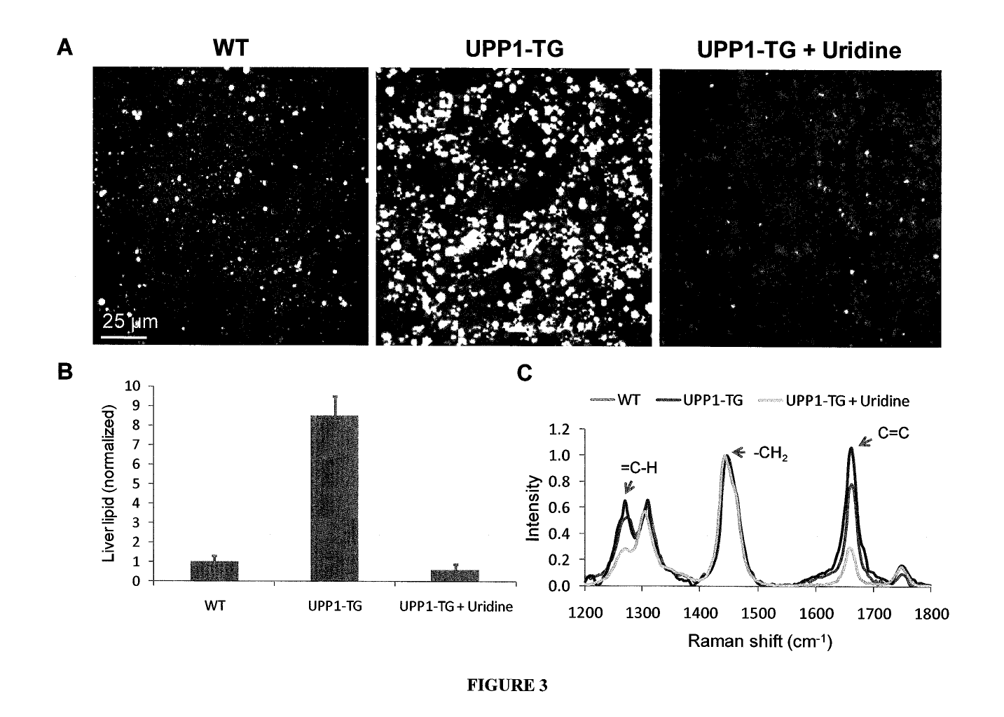

Using CARS imaging, it was found that dietary supplementation with Urd (2 to 5% in dry diet) reduced liver lipid accumulation by nearly 15-fold in UPP-1-TG mice (FIG. 3A-B). Lipid-droplet composition analysis with Raman spectroscopy further revealed the impact of Urd on lipid-chain unsaturation, i.e., the number of carbon-carbon double bond in the lipid chain (FIG. 3B).

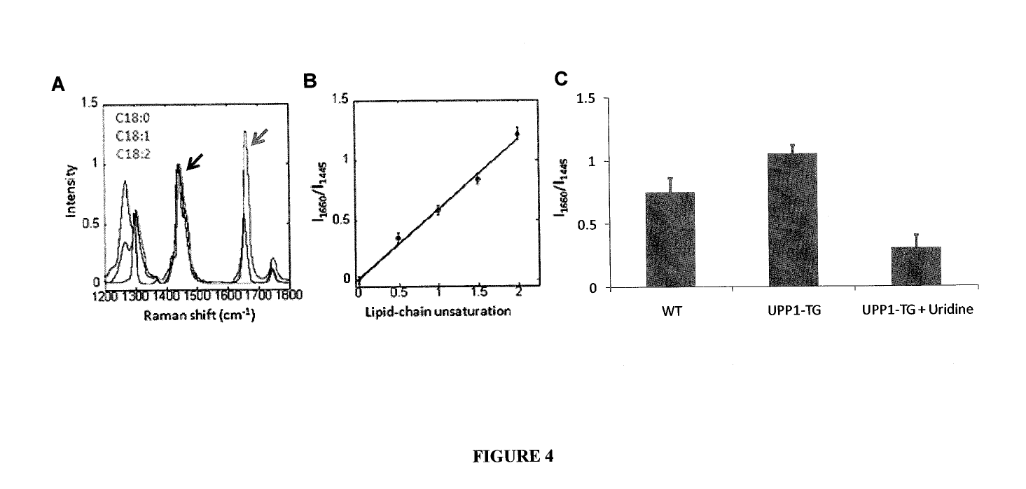

In control UPP-1-TG mice, the value of 11660/11445, which is a reliable measure of lipid-chain unsaturation (FIG. 4A-B; Rinia, H. A., et al (2008) Biophysical Journal 95: 4908-4914, 2008), was 1.05 (FIG. 4C). In UPP-1-TG mice fed with Urd, the 11660/11445 value of liver lipid droplets was 0.3. The 11660/11445 value of liver lipid droplets was 0.8 for control WT mice.

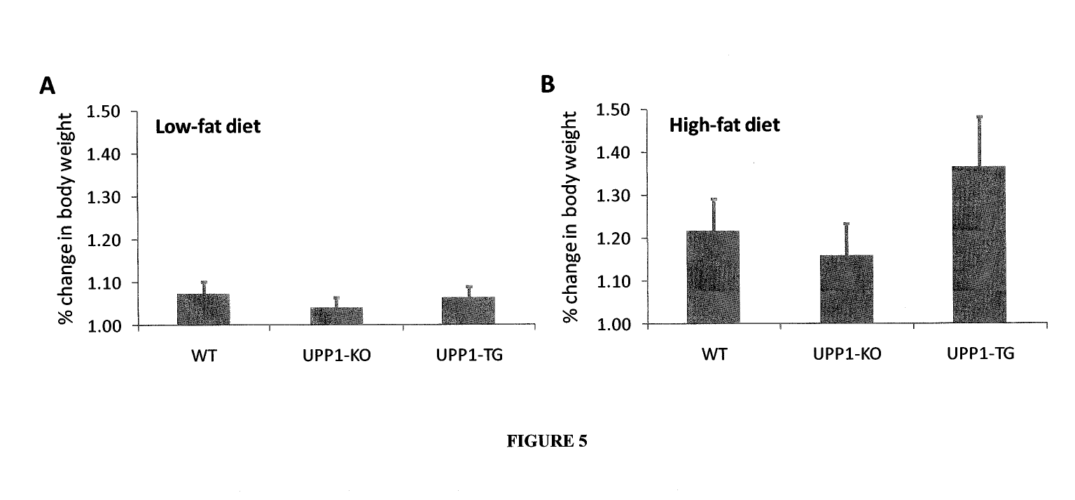

To study the long term effect of different circulating Urd concentrations on lipid accumulation, the three different mouse strains were fed a diet with either 10% of calories derived from fat (Harlan TD.06416, fatty acid profile: 29% saturated, 37% monounsaturated, 34% polyunsaturated) or a high fat diet with 45% of calories from fat (Harlan TD.06415, fatty acid profile: 36% saturated, 47% monounsaturated, 17% polyunsaturated). The data show virtually no difference in weight gain among the WT, UPP-1-KO and UPP-1-TG strains when fed a diet low in calories derived from fat (FIG. 5A). However, in the groups fed the 45% high-fat diet, an obvious difference was observed with the UPP-1-TG mice rapidly gaining more weight than the two other strains. We recorded a 37% increase in weight for the UPP-1-TG compared to 22% in WT and 17% in UPP-1-KO after 4 weeks (FIG. 5B). While the difference in weight between WT and UPP-1-KO was not statistically significant due to the limited sample size (n=6), a lower weight for the UPP-1-KO on both diets was consistently observed. These data confirm previous results indicating that disruption of Urd homeostasis is associated with 1) development of fatty liver, 2) high circulating levels of triglycerides and 3) an obesity phenotype when the UPP-1-TG mice are fed a diet high in calories derived from fat. At the end of the experiment (8-10 weeks) the livers were excised and subjected to histopathological examination for signs of steatosis and progression to steatohepatitis or cirrhosis. General staining with haematoxylin and eosin will confirm basic tissue anatomy, and specialty stains such as trichrome (to evaluate increase of collagen) and periodic acid-Schiff (used to detect glycogen) will be utilized to identify damaged tissue (steatosis progression).

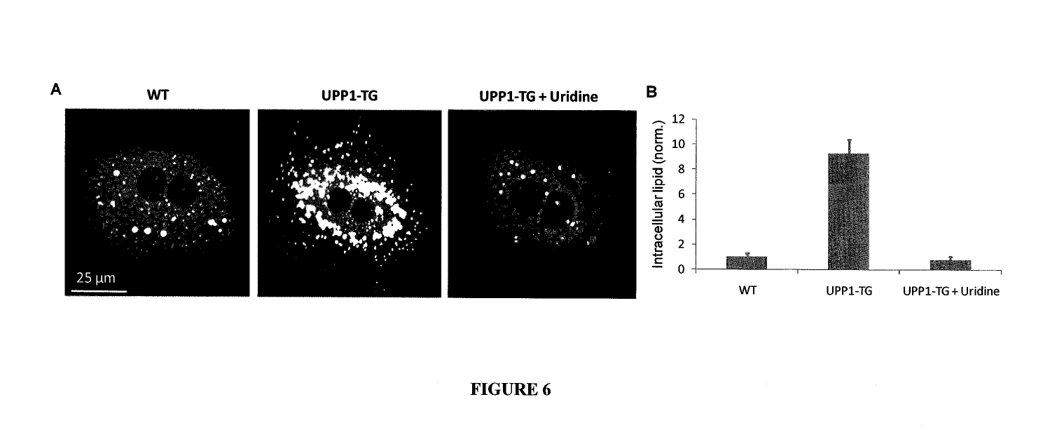

To further confirm our results and to provide a more practical in vitro method, short term cultures of primary liver hepatocytes were generated. WT and UPP-1-TG mouse hepatocytes were isolated using a two-step collagenase perfusion technique initially described by Seglen (Seglen, P.O. (1976) Methods Cell Biol. 13: 29-83). Viability ranged from 80-90% as determined by trypan blue exclusion. Hepatocyte enrichment reached approximately 85-90%. Cells were monitored for albumin expression through 72 hours, with no discernible differences when compared to the initial isolation. Primary hepatocytes from UPP-1-TG mice exhibited a 10-fold reduction in intracellular lipid droplet accumulation after 24 hour incubation with 100 μM Urd (FIG. 6). The observation in primary hepatocytes is consistent with the observation in liver tissues reported in FIG. 3.

### Example 2

### Role of Uridine in the Regulation of Lipid Accumulation in the Liver

Uridine, through its catabolites, contributes directly to the synthesis of fatty acids (de novo lipogenesis). Therefore, high uridine degradation in UPPI-TG mice provides a high quantity of precursors for de novo lipid synthesis leading to increased hepatic lipid accumulation. It was found that the drastic reduction of URD concentration in UPP-1-TG mice compared to WT liver tissue, (0.5 M versus 6.4 respectively) is associated with a significant increase in the tissue concentration of -alanine (186.9 and 80.8 M for UPP-1-TG and WT liver respectively). β-alanine is the final product of the degradation of URD and represents the rate-limiting precursor in the formation of carnosine, an antioxidant able to scavenge reactive oxygen species (ROS) as well as α/β unsaturated aldehydes formed from peroxidation of cell membrane fatty acids during oxidative stress. More importantly, β-alanine is a constituent of acetyl-CoA and malonyl-CoA, therefore directly capable of participating in fatty acids biosynthesis. Also, β-alanine plays an important role as a building block of the growth factor pantothenic acid that is a co-factor in a number of biological reactions, including the synthesis and the catabolism of fatty acids.

The role of URD and its main catabolites (uracil, dihydrouracil, β-alanine and malonate) in the synthesis of fatty acids and triglycerides will be evaluated both in vivo and in primary hepatocytes. Moravek Biochemicals and Radiochemicals (Brea, Calif.) will provide the necessary radiolabeled (³H and ¹⁴C) Urd and catabolites, including compounds to evaluate the role of potential precursors in fatty acid biosynthesis such as: [³H]-β-alanine (MT1527), [2-¹⁴C]-malonic acid (MC312), [6-¹⁴C]-dihydrouracil (MC481), [6-¹⁴C]- or [6-³H]-uracil (MCI59 and MT656), [uracil-¹⁴C(U)]-uridine (MC2313). The effect of URD and its catabolites on fatty acid synthesis will be evaluated in vivo by adding each individual pyrimidine derivative to the standard animal diet (2018 Teklad Global Rodent Diet with 18% of calories from fat) and then measuring the incorporation of [³H]-H₂0 into liver fatty acids. The rates of fatty acid synthesis will be measured in 6-8 week old mice (six per group) during the early light cycle after a 2-h fast. Each animal will be injected intraperitoneally with 50 μCi of [³H]H₂0 in 0.1 ml of saline. One hour after injection, each animal will be anesthetized, and 300-500 μl of blood will be removed from the inferior vena cava and used to measure the plasma [³H]H₂0 specific activity in duplicate. The liver will be removed, 200-300 mg portions of tissue will be saponified, and fatty acids will be extracted from the samples with 10 ml of petroleum ether after acidification with 1 ml of concentrated HCl, followed by a second extraction, evaporation of the petroleum ether and measurement of the incorporated radioactivity (Shimano, H., et al. (1996) J. Clin. Invest. 98:1575-1584).

The ability of URD degradation products to incorporate into the fatty acids of hepatocytes will be determined, utilizing the radiolabeled pyrimidine derivatives previously mentioned. Hepatocytes from WT, UPP-1-KO and UPP-1-TG mice will be prepared as described above (Seglen, P.O. (1976) Methods Cell Biol. 13: 29-83) and resuspended in Krebs-Henseleit buffer containing 1.5% BSA and 10 mM glucose. The hepatocytes (5×10⁶ cells) will be incubated at 37° C. in a shaken water bath in 2.5 mL of Krebs-Henseleit, pH 7.4, containing 1.5% BSA (w/v) and 10 mM of glucose, under an atmosphere of carbogen (95% C0₂; 5% 0₂) (Carrasco, M. P., et al. (1998) Biochem. Pharm. 56: 1639-1644). The reactions will initiate after 90-min incubation by adding the pyrimidine catabolites at different concentrations from 5-200 μM mixing the cold substrates with 5-25 μCi of the corresponding radiolabeled derivatives. The reactions will be continued for 120 minutes at 37° C. and stopped by the addition of 7.5 mL ice-cold Krebs-Henseleit. The cells will then be washed twice in Krebs-Henseleit medium at 50 g for 5 minutes and the pellet collected for the analysis of lipids. Lipids will be extracted from the cell pellet according to the procedure of Folch (Folch J., et al. (1957) J. Biol. Chem. 226: 497-509). Free fatty acids and triglycerides will be separated on silica gel 60 G TLC plates (Merck) pretreated with hexane, developed initially with hexane:benzene (1:1) then followed by a mixture of hexane/diethyl ether/acetic acid (80:20:1). The plates will be sprayed with a CuS0₄ solution and lipids visualized by heating at 180° C. for 15 minutes, then scraped and transferred to scintillation vials for radioactivity measurements (Saint-Leger, D. and Bague, A. (1981) Archives of Dermatological Research 271: 215-222).

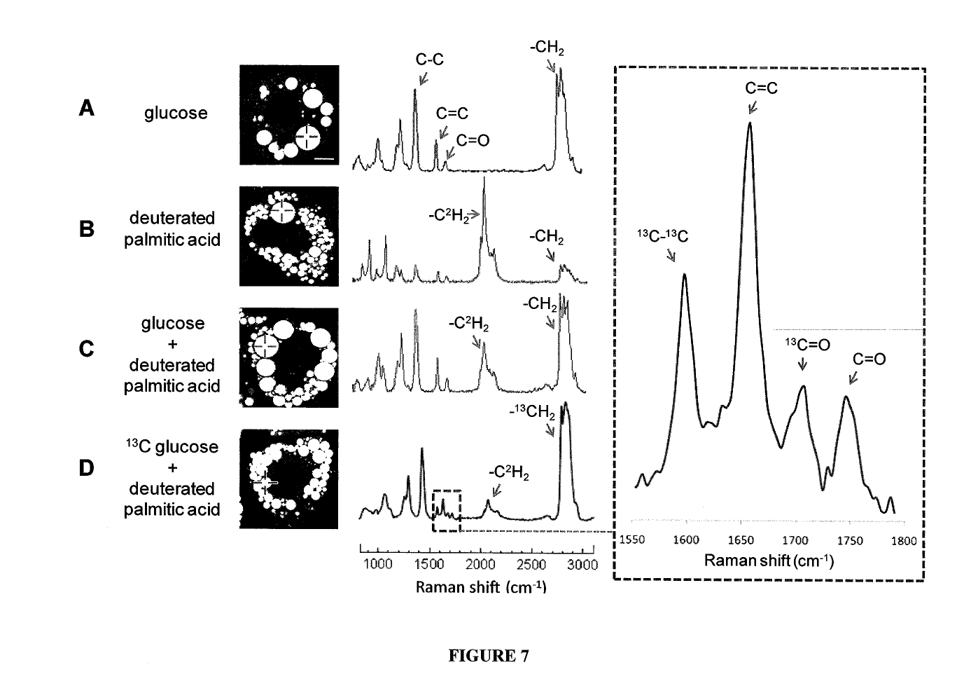

As an alternative to the previous methodology, CARS microscopy will be used to exploit the difference in the vibrational frequencies between ¹²C-¹²Cand ¹³C-¹³C or ¹²C-¹H and ¹²C-²H to selectively monitor the trafficking of ¹³C or ²H-labeled molecules. FIG. 7 provides examples of how CARS imaging coupled with Raman spectroscopy can study the contribution of deuterated (³H) palmitic acids and/or ¹³C glucose to lipid droplet composition in 3T3-L1 cells undergoing fat-cell differentiation. Similar applications in primary hepatocytes will allow tracking of the contribution of deuterated or 13C labeled Urd, Urd catabolites, and other metabolites to liver lipid metabolism.

It is believed that the high rate of uridine degradation in UPP-1-TG mice results in unbalanced deoxynucleotide pools causing mitochondrial DNA (mt DNA) instability and impairment of the mitochondrial respiratory chain. Disruption of mitochondrial biogenesis and P-oxidation of fatty acids lead to hepatic lipid accumulation. Generally, abnormalities in mitochondrial P-oxidation of fatty acids lead to microvesicular hepatic steatosis (Jaeschke, H., et al. (2002) Toxicol. Sci. 65: 166-176. Fatty acid P-oxidation occurs in both mitochondria and peroxisomes. However, mitochondria catalyze the P-oxidation of the bulk of short-, medium-, and long-chain fatty acids derived from diet and this pathway constitutes the major process by which fatty acids are oxidized to generate energy (Reddy, J. K. and Rao, M. S. (2006) Amer. J. Phys. 290: G852-G858).

Several dideoxynucleoside analogs utilized as antiviral agents to treat patients with human immunodeficiency virus (HIV) infection have been shown to decrease mitochondrial DNA (mtDNA) leading to an acquired equivalent of a mitochondrial cytopathy. DNA polymerase-y, which is found in mitochondria, incorporates dideoxynucleoside triphosphates into the growing chain of DNA, thus impairing mtDNA replication (Simpson, M. V., et al. (1989) Biochem. Pharmacol. 38: 1033-1036), leading to reduced quantities of mtDNA and consequent mitochondrial problems. Inherited and acquired mitochondrial cytopathies are the result of inadequate energy production.

A mouse lacking both UPP-1 and thymidine phosphorylase activity (UPP-1-KO/TP-KO) has been developed, resulting in elevations of the pyrimidine nucleosides Urd, thymidine (Thd) and deoxyuridine (dUrd) in plasma and tissues which mimics the characteristics of mitochondrial neurogastrointestinal encephalopathy (MNGIE) (Lopez, L. C., et al. (2009) Human Mol. Gen. 18: 714-722, 2009). Significant increases of dTTP were detected in brain and liver mitochondria and a significant decrease of dCTP in brain mitochondria of UPP-1-KO/TP-KO mice. A 27% reduction in mtDNA was observed in the brain of 6-month-old UPP-1-KO/TP-KO mice using quantitative real-time PCR of COX1 (mtDNA) and GADPH (nDNA). The reduction of mtDNA was even more pronounced in older mice (14-18 months old), which showed 61% depletion of mtDNA in brain relative to WT mice, as confirmed by Southern blot analysis (Lopez, L. C., et al. (2009) Human Mol. Gen. 18: 714-722, 2009). Diminished levels of mtDNA-encoded proteins and decreased mitochondrial respiratory chain function were observed in the brain of UPP-1-KO/TP-KO mice. The effect on mtDNA in liver was much less severe. It is believed that the severe alteration in Urd concentration in UPPI-TG mice may lead to unbalanced deoxynucleotide pools causing mt DNA instability and impairment of mitochondrial respiration chain function in hepatocytes. Mitochondrial-oxidation of fatty acids generates acetyl-CoA and reducing equivalents (NADH and FADH2), which are linked to the Krebs cycle and the mitochondrial respiratory chain, leading to ATP production in aerobic tissues. Altering Urd concentrations in tissues either by knocking-out or knocking-in UPP-1 may result in similar mitochondrial disruption. Notably, in the UPPI-KO mouse, the alterations in Urd concentrations are not as dramatic as observed in the UPP-1-TG, which has a 10-fold Urd reduction in plasma and liver concentrations. Furthermore, the administration of exogenous Urd is believed to result in a temporary modification of the circulating Urd and tissue levels with minimal disruption of the ribo- and deoxyribo-nucleotide pools.

Mitochondrial deoxynucleotide (dNTP) pools by the DNA polymerase extension assay will be measured as described previously (Ferraro, P., et al. (2006) Proc. Natl. Acad. Sci. USA, 103: 18586-18591; Song, S., et al. (2005) Proc. Natl. Acad. Sci. USA, 102: 4990-4995). Briefly, liver homogenate will be centrifuged at 1000 g for 3 minutes at 4° C. (twice) and supernatants will be centrifuged at 9000 g for 5 min at 4° C. (twice). Mitochondrial pellets from liver will be re-suspended in 200 μl of cold water and an aliquot of 10 μl will be used to measure proteins. Then, the dNTPs will be extracted with 60% methanol and after evaporation the dry residue re-suspended in 60 μl of water (Ferraro, P., et al. (2006) Proc. Natl. Acad. Sci. USA, 103: 18586-18591; Song, S., et al. (2005) Proc. Natl. Acad. Sci. USA, 102: 4990-4995). To measure the dNTPs pool, 2 μM of [³H]-dATP or [³H]-dTTP will be used in each reaction. Mouse mtDNA will be quantitated by real-time PCR using an ABI PRISM 7000 sequence detection system as described using primers and probes for murine COX 1 gene (mtDNA) and mouse glyceraldehyde-3-phosphate dehydrogenase (nDNA) (Spinazzola, A., et al. (2006) Nat. Genet., 38: 570-575). The values of mtDNA levels will be normalized by nDNA, and the data expressed in terms of percent relative to WT mice.

Rates of β-oxidation will be determined by incubating mitochondria at 30° C. with [1-¹⁴C]palmitoyl-L-carnitine or [1-¹⁴C]palmitoyl-CoA+1 mM L-carnitine (Madsen, L., et al. (1999) Biochem. Pharmacol. 57, 1011-1019). Rates of oxidation in primary hepatocytes will be measured as for isolated mitochondria (above) except that [1-¹⁴C]palmitic acid will be used as substrate. Production of acid-soluble radioactivity from [1-¹⁴C] fatty acid is given as nanomoles of fatty acid consumed/h/10⁶ cells at 30° C. The reaction will be quenched with perchloric acid 10 minutes after addition of substrate. The reactions will be extracted with hexane and acid precipitable material will be counted using a scintillation detector.

Oxygen consumption in freshly prepared mitochondria will be measured polarographically with a Clark-type electrode (Oxigraph Hansatech) after the addition of glutamate and malate (G/M) at 5 mM or succinate (SC) at 5 mM with rotenone at 1 μg/ml followed by ADP at 0.3 mM. Mitochondria will be then uncoupled by the addition of 2,4-dinitrophenol to a final concentration of 25 μM. The respiratory control ratio (RCR) will be calculated by dividing the state III by state IV (post ADP depletion) oxygen consumption rates (Gnaiger, E. (2001) Respir, Physiol. 128: 277-297; Gnaiger, E. and Kuznetsov, A. V. (2002) Biochem. Soc. Trans. 30: 252-258). The activities of citrate synthase and complexes I, II, III, IV, and II+III will be measured spectrophotometrically at 37° C. in the isolated mitochondria through modified procedures of Malgat et al. (Malgat, M., et al. (1999) Enzymatic and polarographic measurements of the respiratory chain complexes. In: Mitochondrial Diseases: Models and Methods, edited by Lestienne P. Paris: Springer-Verlag, p. 357-377).

### Example 3

### Roles of UPP-1 and UPP-2 in Lipid Regulation

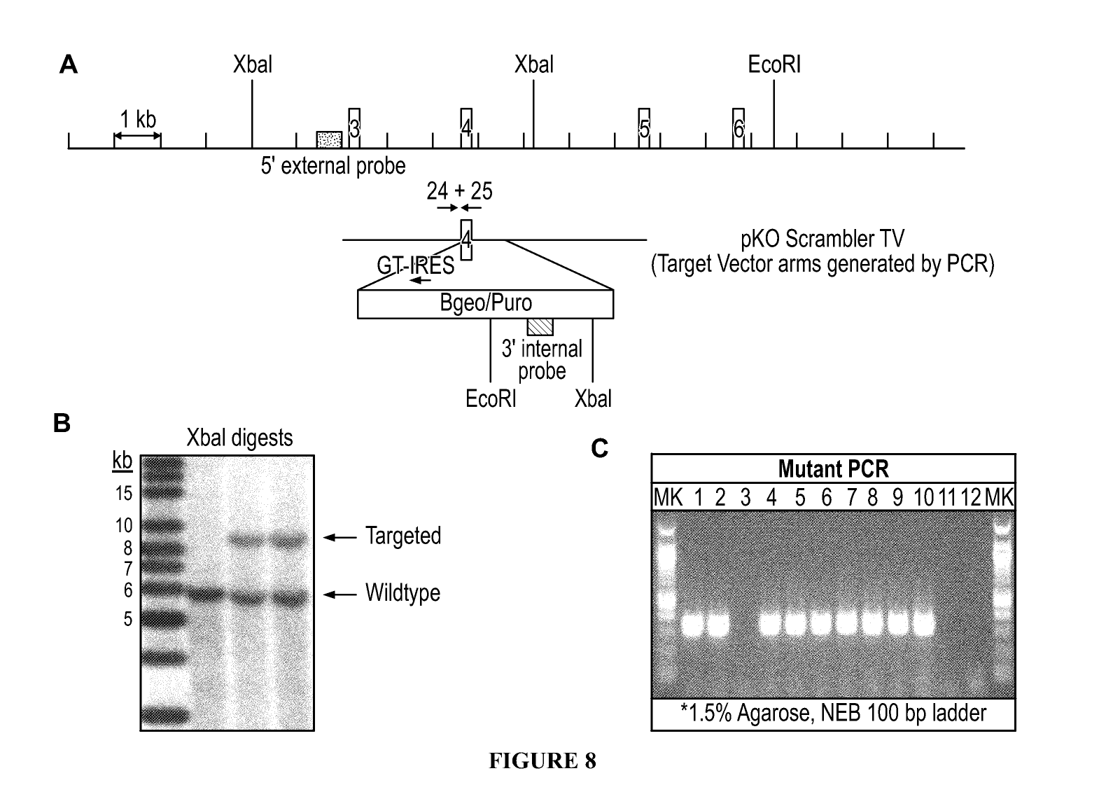

The creation of a UPP-2-KO mouse model has been initiated by generating a construct with a targeted insertion which deletes 800 bp of the UPP-2 gene, including all of exon 4 (FIG. 8). ES cell screening has demonstrated successful targeting at the UPP-2 locus by both Southern blot and PCR analysis (FIG. 8). The first progeny from the chimeric parent mouse has been received and will be bred to homozygosity before the analysis of the effect on Urd homeostasis and liver lipid accumulation. This UPP-2-KO model will be subsequently bred with the already established UPP-1-KO to create an animal completely deficient in uridine phosphorylases.

The preparation of a construct to obtain a UPP-2 transgenic mouse model has also been initiated, in order to completely characterize the specific role UPP-2 plays in Urd metabolism and subsequent fatty acid metabolism. To generate a conditional UPP-2 transgenic animal model, the same targeting strategy that we used to successfully create the UPP-1-TG mouse model will be utilized. The UPP-2 coding sequence will be synthesized and cloned into the ROSA26 targeting vector by Genscript (Piscataway, N.J.). ES cell transfection and chimeric mouse generation will be completed by the Yale Animal Genomics Services (YAGS). The final homozygous knock-in mouse will be crossed with mice expressing Cre-recombinase in liver, or ubiquitously if warranted by preliminary data. Ultimately, the UPP-2-KO model will be bred with the UPP-1-KO animal to create an animal completely deficient of URD phosphorolytic activity. By developing conditional knock-ins and knock-outs, tissue-specific roles of the phosphorylases can be evaluated.

### Example 4

### Structure and Function of UPP-2

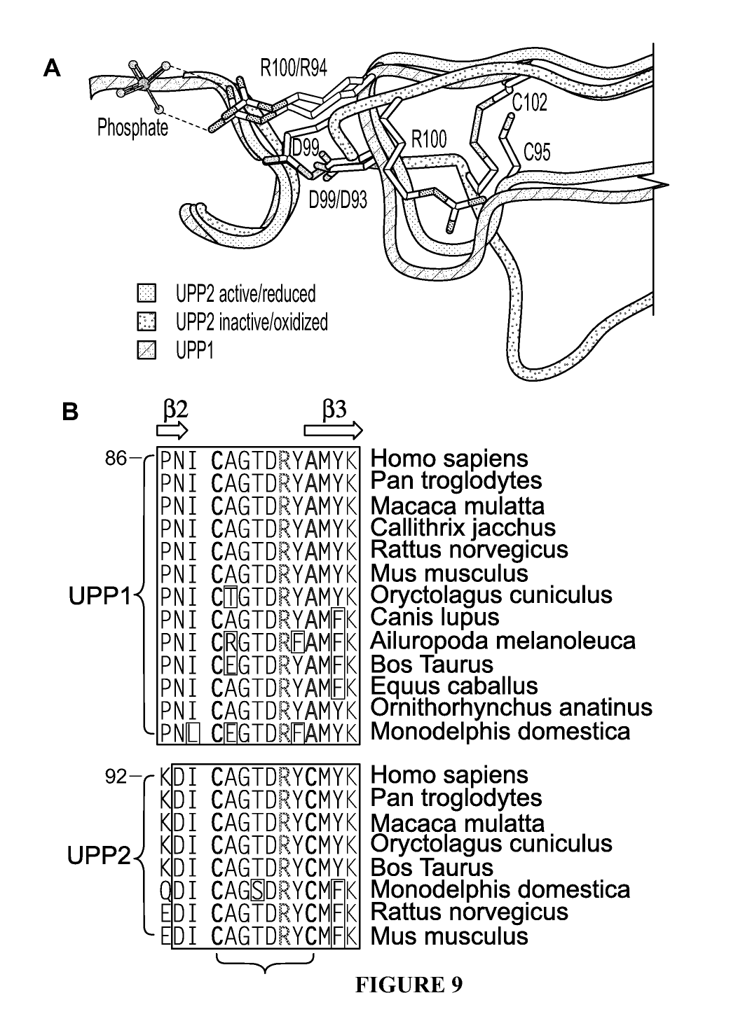

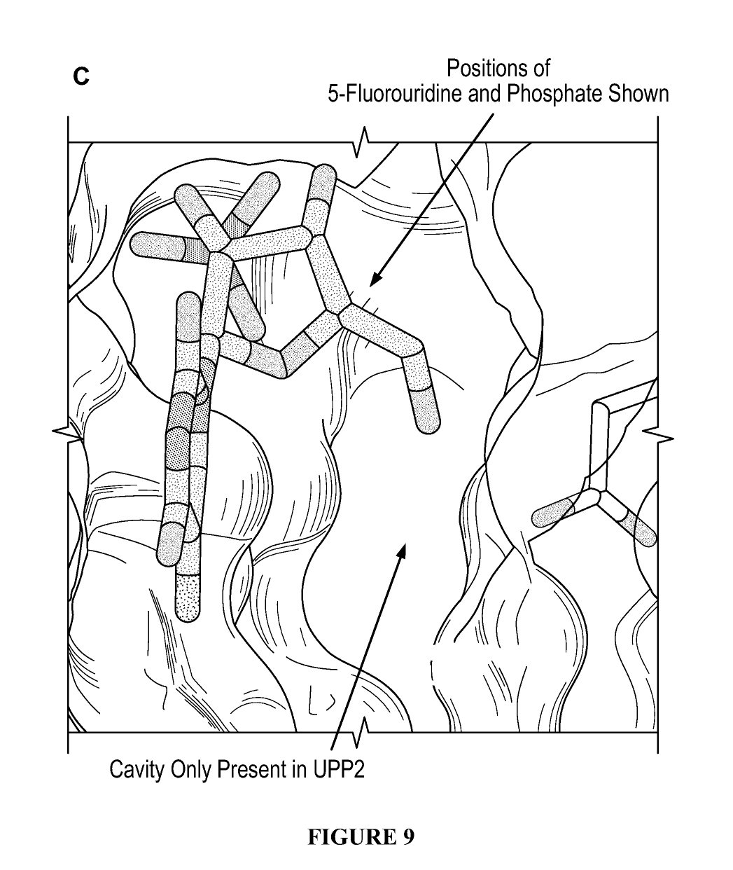

Crystallographic structure determination of UPP-2 in two distinct conformations was performed, having collected 1.5 Å & 2.0 Å datasets at SSRL and phased the data using Molecular Replacement, and searching with a homology model of UPP-2 constructed from the known structure of UPP-1 (Roosild, T. P., et al. (2009) BMC Struct. Biol. 9: 14-17). These high resolution structures, revealed unequivocally the presence of an intramolecular disulfide bridge that repositions a critical, active-site, phosphate-coordinating arginine residue, Arg100, to a location that does not support catalysis of the enzyme's phosphorolytic activity (FIG. 9, A-B). Consistent with this structural finding, in vitro comparison of the activity of murine UPP-1 and UPP-2 activity reveals a substantial sensitivity to oxidative inactivation in the latter homologue. Together, these results demonstrate that UPP-2 may possess an intrinsic mechanism for inactivation in the presence of oxidative conditions and may be a molecular target of ROS signaling.

Pilot experiments have shown that UPP-2 is much more sensitive than UPP-1 to inactivation by dissolved oxygen and oxidized glutathione. This inactivation appears to be fully reversible by both DTT and reduced glutathione. Specific activity measurements on exposure to various ROS and other known biologically important oxidants will be performed. A full characterization of the enzyme kinetics of UPP-2 will be conducted utilizing the absorbance change (A280) accompanying the conversion of uridine to uracil, as has been done previously for UPP-1 (Renck, D., et al. (2010) Arch Biochem Biophys 497: 35-42). Site-directed mutagenesis of UPP-2 (C102A) will be used to validate the importance of the observed disulfide bridge to the solution behavior and function of this enzyme and will be a control to distinguish specific, directed inactivation from indiscriminate protein-damage by oxidative compounds. All proteins will be prepared using the recombinant expression and purification methods that enabled their structure determination (Roosild, T. P., et al. (2009) BMC Struct. Biol. 9:14-17).

Numerous pathways that control the gene expression of UPP-1 have been identified, including an inhibitory role for p53 (Zhang, D., et al. (2001) Cancer Res. 61: 6899-6905). Future experiments will evaluate how the UPP-2 promoter is regulated in comparison to UPP-1, such as, e.g., via transcription factors that are linked to lipogenesis or -oxidation. The promoter region of the murine UPP-2 gene will be characterized and the potential regulatory elements that control UPP-2 transcription will be evaluated. The genomic clone RP23-149P17 from the RPCI-23 BAC Library (derived from 5-week old female C57BL/6 mice) has been thoroughly sequenced and shown to contain the 5′ end of the murine UPP-2 gene (accession number AL732468). A luciferase expression vector containing 4,000 base pairs upstream of the initiator methionine, including the 5′UTR and proximal promoter elements, has been cloned. Several 5′ deletions of this luciferase construct will be generated and transfected into murine AML12 cells (nontransformed hepatocytes derived from mice transgenic for TGF-α; Wu, J. C., et al. (1994) Proc. Natl. Acad. Sci. USA 91: 674-6) or isolated primary hepatocytes. To confirm the activity of factors that potentially may mediate transcriptional regulation, the deletions (or site-directed mutants) will be tested for their sensitivity to transcriptional regulators. To complete the elucidation of UPP-2 gene regulation by the identified transcriptional regulators, gel mobility shift assays (EMSA) and footprinting analysis will be performed.

Structural analysis of UPP-2 shows that the formation of the disulfide bridge is accompanied by substantial conformational changes in the surface character along one face of the enzyme that is likely to form a protein-protein interface. The hypothesis that UPP-2's redox state affects its interactions with other potentially regulatory and/or signaling protein subunits will be examined by using a benzylacyclouridine (BAU) affinity column to pull down in vivo complexes of UPP-2 from UPP-1-KO mouse livers under aerobic and anaerobic conditions, following the methods used previously to analyze UPP-1 (Russell, R. L., et al. (2001) J. Biol. Chem. 276: 13302-13307). 5′-NH2-BAU, an inhibitor of UPP-1 and UPP-2, will be used to prepare an affinity column using an Affigel-10 matrix (Bio-Rad, Hercules, Calif.). After initial purification on a DEAE column, the Tris-extract from UPP-1-KO mouse liver will be applied to the 5′-NH₂-BAU affinity column. UPP-2 bound to the column will be eluted with 20 mM Urd. The eluate containing the murine enzyme will be then dialyzed against 20 mM Tris buffer (pH 7.5) and concentrated using Centricon-10 microconcentrators (Amicon, Beverly, Mass.), and sequenced using an Orbitrap mass spectrometer available through the NVCI Proteomics Core facility.

Another consequence of the redox controlled structural changes in UPP-2 is that the alternate conformation creates a cavity adjacent to the active site that has not been previously observed in any other uridine phosphorylase structures (FIG. 9C). This finding provides evidence that certain uridine analogs, with expanded molecular structure extending beyond the ribose sugar group, can selectively discriminate UPP-2 from UPP-1 by taking advantage of the novel flexibility of the former enzyme. A 1338-member library of uridine derivatives has been developed (Hang, H. C., et al. (2004) Chem. & Biol. 11: 337-345). These compounds are functionalized at the 5′ position of the ribose sugar using 446 aldehydes connected via an oxime or hydrazone linkage, creating a diverse array of molecules that extend from a uridine-like scaffold, several of which we have been able to model fitting into the void observed in the inactive UPP-2 structure. This library will be assayed in 96-well format for each compound's ability to inhibit uridine phosphorolysis by either UPP-1 or UPP-2, as indicated by A280 stability, to identify selective inhibitors of UPP-2. Any lead compounds identified in the assay will be enhanced through synthesis of their 5-benzyl-uracil derivatives, as tins moiety has been shown to substantially increase the affinity of small molecules to uridine phosphorylases in general, and improve their specificity to these enzymes over other uridine binding proteins (Chu, M. Y., et al. (1984) Cancer Res. 44: 1852-1856).

Although the invention has been described with reference to the above examples, it will be understood that modifications and variations are encompassed within the spirit and scope of the invention. Accordingly, the invention is limited only by the following claims.

1. A method of treating a fatty liver disorder in a subject, comprising administering to the subject a therapeutically effective amount of a uridine phosphorylase (UPP) modulator.

2. The method of claim 1, wherein the uridine phosphorylase is UPP-1.

3. The method of claim 1, wherein the uridine phosphorylase is UPP-2.

4. The method of claim 1, wherein the UPP modulator is uridine.

5. The method of claim 1, wherein the UPP modulator is a pharmaceutically acceptable salt of uridine.

6. The method of claim 1, wherein the UPP modulator is capable of modulating both UPP-1 and UPP-2.

7. The method of claim 1, wherein the UPP modulator is capable of modulating UPP-1 without modulating UPP-2.

8. The method of claim 1, wherein the UPP modulator is capable of modulating UPP-2 without modulating UPP-1.

9. The method of claim 1, wherein the UPP modulator is a small molecule compound.

10. The method of claim 1, wherein the UPP modulator is an antibody, protein, or polypeptide.

11. The method of claim 1, wherein the UPP modulator is a polysaccharide.

12. The method of claim 1, wherein the UPP modulator is a nucleic acid.

13. The method of claim 1, wherein the UPP modulator is carried within food when administered to the patient.

14. A pharmaceutical composition comprising a therapeutically effective amount of a uridine phosphorylase (UPP) modulator and a pharmaceutically acceptable carrier or diluent.

15. The pharmaceutical composition of claim 14, wherein the UPP modulator is uridine.

16. The pharmaceutical composition of claim 14, wherein the UPP modulator is a pharmaceutically acceptable salt of uridine.

17. The method of claim 14, wherein the UPP modulator is a small molecule compound.

18. The method of claim 14, wherein the UPP modulator is an antibody, protein, or polypeptide.

19. The method of claim 14, wherein the UPP modulator is a polysaccharide.

20. The method of claim 14, wherein the UPP modulator is a nucleic acid.
